(19) United States
(12) Patent Application Publication
(10) Pub. No.: US 2023/0284611 A1
(43) Pub. Date: Sep. 14, 2023

(54) AQUEOUS SOLUTION TO PRESERVE NUCLEIC ACIDS

(71) Applicant: Biofarma S.r.l., Mereto Di Tomba (IT)

(72) Inventors: Emanuele NENCIONI, San Daniele Del Friuli (IT); Michela BULFONI, San Vito Al Tagliamento (IT)

(21) Appl. No.: 18/018,490

(22) PCT Filed: Jul. 1, 2021
(86) PCT No.: PCT/IT2021/050205

§ 371 Date: Jan. 27, 2023

(30) Foreign Application Priority Data
Jul. 30, 2020 (IT) 102020000018682

(51) Int. Cl. A01N 1/02; G01N 1/30; C12Q 1/70

(52) U.S. Cl. CPC A01N 1/021; C12Q 1/701; G01N 1/30

(57) ABSTRACT

An aqueous solution suitable to maintain the integrity of nucleic acids present in a biological sample for subsequent molecular biology analyzes or for medium (room temperature) or long-term (freezing) storage of the biological sample taken is provided.

### FIELD OF THE INVENTION

Embodiments described here concern an aqueous solution to preserve nucleic acids for the purpose of subsequent molecular biology analyses.

### BACKGROUND OF THE INVENTION

Molecular diagnostics comprises the methodologies necessary to carry out laboratory diagnoses, both qualitative and quantitative, based on nucleic acids.

The pre-analytical phase that precedes the actual analysis comprises a series of procedures intended for the maximum recovery of the nucleic acids originally present in the biological sample at the time of collection, therefore, the suitability of the entire pre-analytical phase translates into greater diagnostic quality.

Of fundamental importance are:
transport of the sample, which must be carried out in suitable sterile containers,
speed of transport of the sample, since the degradation of nucleic acids is a function of time,
adequacy of the preservation medium, in order to preserve the characteristics of the biological sample taken,
controlled temperature, generally at + 4° C., occasionally at room temperature.

These procedures can be particularly important in the event that the biological sample is taken with the aim of detecting microorganisms, such as bacteria or viruses. In these cases, in fact, it is necessary to prevent the alteration, both quantitative and qualitative, of microbial facies before molecular analysis.

The swab is a microbiological test with minimum invasiveness, performed in various anatomical sites, in which pathogenic microorganisms and typically commensal resident flora coexist. The most commonly performed swabs are nasal, pharyngeal, ocular, urethral, skin, genital and rectal swabs.

For example, for the diagnosis of upper respiratory tract infections, such as for the recent Coronavirus Disease 19 (COVID-19) pandemic caused by Sars-CoV-2, the oral-pharyngeal and nasal swab is the gold standard procedure.

The collection of this type of sample, mainly from nasopharyngeal respiratory swabs, poses some challenges that include the need for specialized personnel, the risk of exposure of healthcare workers, invasiveness and discomfort for the patient, low sensitivity and variability of the operator/employee that takes the sample.

To overcome these limits, other types of biological matrices are being investigated. Among these, saliva is causing considerable interest due to the ease of collection and the homogeneity of sampling.

Given the practicality of the use of saliva, especially in some categories of subjects, such as children, the elderly, uncooperative people, there is a need to use transport solutions that solve these problems in order to increase the number of tests performed. Numerous methods for collecting saliva have been tested with the aim of optimizing this type of sample.

Studies on the use of saliva for the detection of SARS-CoV-2, by means of RT-PCR, have been increasing recently, showing saliva as a very attractive, readily available and repeatable biological matrix.

One of the problems to be solved, in particular, is that, if not properly preserved, the microorganisms degrade or die quickly in a dry environment and at some concentrations of atmospheric oxygen.

It is known to use aqueous solutions, also defined as preservation media, which allow the survival of the microorganisms contained in the biological sample during transport.

These media are designed to maintain the vitality of the microorganisms, therefore the biological sample preserved in these aqueous solutions must be analyzed quickly.

The biological samples preserved in these media must be sent immediately to the laboratory or alternatively they can be preserved at 4° C. for no longer than 48-72 hours, after which the degradation of the viruses begins, especially due to the instability of the viral RNA. Furthermore, it is possible that these media unwantedly promote the growth of the microorganisms that make up the commensal flora, taken during the sampling, influencing the correct identification of the target microorganism.

To obviate this, it is possible to add to the medium stabilizers of the nucleic acids (guanidinium hydrochloride, isothiocyanate), β-mercaptoethanol, detergents, nutrients, cryopreservatives (DMSO), antibiotics and antifungals which however have the disadvantage of being harmful for those who have to handle the medium which therefore needs to be used following precise safety rules.

In general, as with the current COVID-19 infection, the media Universal Transport Medium (UTM® - COPAN) or Virocult® (ALIFAX) are the most commonly used to immerse the swab with the biological sample and preserve the viruses in it, possibly contained.

UTM®, for example, comprises an aqueous solution of salts, gelatin and bovine serum albumin (BSA) as stabilizers, sucrose, glutamic acid and HEPES. The presence of buffered salts in the medium protects microorganisms sensitive to pH variations. Gelatin and BSA are a source of nourishment to support vitality during possible preservation and transport. Sucrose helps to preserve the microorganisms when the sample is frozen. There is also an antibiotic to inhibit bacterial growth.

Alternative media can be made, if necessary, by adding for example PBS (Phosphate-Buffered Saline) or HBSS (Hank’s Balanced Salt Solution) at pH 7.2 glucose (1 g/L), lactalbumin (3 g/L), chloramphenicol (2.5 µg/ml) and cyclohexoxymide (10 µg/ml) as an antifungal agent, to a phosphate buffer or other similar formulations.

When dedicated media is not available or there is no access to or availability of the components to make one as needed, the Center for Disease Control and Prevention (CDC) and the Food and Drug Administration (FDA) recommend the use of phosphate buffer alone in order to collect and preserve the samples.

By examining the compositions of the media as above, it is understandable that a medium rich in substances performs better in terms of preservation power compared with the simple phosphate buffer, but on the other hand it is more expensive and difficult to produce.

Document WO-A-00/06780 describes the preparation of a preservation medium for RNA known as RNAlater®. In example 2 of this document, we describe making available 0.5 M EDTA disodium dihydrate (18.61 g/100 ml, pH at 8.0 with NaOH stirred), 1 M of sodium citrate trisodium salt dihydrate (29.4 g/100 ml), ammonium sulfate powder and sterile water. In particular, 40 ml of 0.5 M EDTA, 25 ml of 1 M sodium citrate, 700 g of ammonium sulfate and 935 ml of sterile distilled water are combined in a beaker, stirring everything until the ammonium sulfate is dissolved. After cooling, the pH is adjusted to pH 5.2 by adding 1 M H2SO4. Consequently, the aqueous solution thus prepared has an overall weight of 1700 g and contains, in weight/weight percentage, 0.437% w/w of EDTA disodium dihydrate, 0.432% w/w of sodium citrate trisodium salt dihydrate and 41.18% w/w of ammonium sulfate.

Document CN-A-111088319 describes a preservative solution for RNA samples which includes sodium citrate, proteinase K, sulfuric acid, EDTA and ammonium sulfate. The sulfuric acid is present to adjust the pH between 4.5 and 5.8.

Both the preservation medium known from WO-A-00/06780 and also the preservative solution known from CN-A-111088319 also have the disadvantage that they require a pH adjustment. Moreover, both are only usable for RNA and not for DNA.

There is therefore a need to perfect an aqueous solution for preserving nucleic acids which can overcome at least one of the disadvantages of the state of the art.

In particular, one purpose of the present invention is to provide an aqueous solution which allows to preserve, in a stable way over time, nucleic acids such as DNA and RNA.

Another purpose of the present invention is to provide an aqueous solution compatible with molecular biology techniques.

Another purpose of the present invention is to provide an aqueous solution suitable to preserve bacterial, viral or cellular DNA or RNA.

It is also a purpose of the present invention to provide an aqueous solution which inhibits bacterial or viral proliferation.

Another purpose of the present invention is to provide an aqueous solution which is simple and inexpensive to produce.

The Applicant has devised, tested and embodied the present invention to overcome the shortcomings of the state of the art and to obtain these and other purposes and advantages.

### SUMMARY OF THE INVENTION

The present invention is set forth and characterized in the independent claims. The dependent claims describe other characteristics of the present invention or variants to the main inventive idea.

In accordance with the above purposes, an aqueous solution is provided to stably preserve nucleic acids in a biological sample, which overcomes the limits of the state of the art and eliminates the defects present therein.

The aqueous solution comprises:
an EDTA-based compound between 0.70 and 0.78% w/w with respect to the total weight of the solution;
ammonium sulphate between 17.5 and 38.5% w/w with respect to the total weight of the solution;
a sodium citrate based compound between 0.70 and 0.77% w/w with respect to the total weight of the solution.

The invention provides an aqueous solution, in particular a saline aqueous solution, cheap and easy to prepare, suitable to maintain the integrity of the sample for subsequent molecular biology analyzes or for medium storage (room temperature) or long-term storage (freezing) of the biological sample taken.

Many biological samples are taken in places where there are no facilities to carry out molecular analysis, therefore, these samples have to be transported to dedicated analysis laboratories.

In this context, preserving nucleic acids, such as DNA and RNA, of high quality and quantity is fundamental. DNA and RNA in particular degrade as time passes and temperature increases.

The best-known way to preserve RNA is to freeze the samples in liquid nitrogen, followed by preservation at -80° C. which, however, can be difficult if not impossible to accomplish. Commercial stabilized buffers such as RNAlater (Qiagen; Invitrogen; Zymo research) can preserve RNA even at room temperature. The aqueous solution provided by the present invention, instead of preserving the vitality of the microorganism of interest, provides to stabilize it by inactivating it and keeping it unchanged.

In this way, viruses and bacteria present, such as the commensal flora, are fixed in a stable manner: the nucleic acids will not undergo changes and the resident bacteria will be unable to proliferate interfering with the analyzes downstream.

As known, biological samples are subject to the action of RNases (Ribonucleases) released by lysed cells and by RNases present in the environment, which can rapidly degrade RNA.

The use of an aqueous solution with acid pH and with high concentrations of salts allows the inactivation and denaturation of these harmful enzymes, while the high ionic charge stabilizes the structure of the nucleic acids.

In the formulation, a surfactant can be advantageously added to promote the lysis of the cell membranes and the denaturation of the nucleases.

In preferred embodiments, the surfactant can be an anionic surfactant, preferably Sodium Dodecyl Sulfate (SDS), at low concentrations.

The inactivation effect on the microorganisms does not require the addition of antibiotics or antifungals and the absence of this harmful substances guarantees greater safety for the operator.

The aqueous solution described here does not subject the molecular analysis of the biological sample to stringent time limits, since it can be done even up to 48 hours from sample collection, in the case of preservation at room temperature, or even up to 7 days after sample collection, in the case of preservation by freezing at -20° C. It is therefore possible to significantly reduce the number of trips required to transport the samples from the collection site to the analysis laboratory.

Since there are no magnesium, calcium (chelated by EDTA), chloride, phenol ions or other contaminants, the aqueous solution is compatible with all methods of extraction and analysis by means of PCR, real-time qPCR and sequencing, including new generation ones.

The quality of nucleic acids recovered allows to obtain quality gene material for high-throughput investigations.

The aqueous solution is suitable for long-term preservation of biological samples, in ideal freezing conditions, allowing to create biobanks of biological samples intended for carrying out clinical activities or used for research purposes.

Since it is an aqueous solution substantially comprising salts, it can be advantageously sterilized by means of irradiation, for example of gamma rays, without causing degradation of the components in solution.

The invention also provides a dry powder composition which comprises at least:
the EDTA-based compound between 1.9 and 3.8% w/w with respect to the total weight of the composition;
ammonium sulfate between 92.5 and 96.2% w/w with respect to the total weight of the composition;
the sodium citrate based compound between 1.9 and 3.7% w/w with respect to the total weight of the composition.

The dry composition thus provided has to be dissolved in a suitable quantity of solvent, preferably deionized water, in order to reconstitute the aqueous solution in liquid form, in particular saline aqueous solution, with the concentration characteristics described above.

Some embodiments also concern a method to preserve nucleic acids comprising making available a liquid aqueous solution in accordance with the present description or a dry powder composition in accordance with the present description, suitably dissolved in a solvent, and introducing and keeping immersed a biological sample in the liquid aqueous solution or in the solution obtained by dissolving the dry powder composition as above in the solvent.

In possible implementations, the nucleic acids as above are supplied by means of a swab.

In particular, a possible advantageous implementation is the collection and preservation of a salivary sample.

Advantageously, the solution as above in accordance with the present description or the liquid solution obtained by dissolving the dry powder composition in accordance with the present description in the solvent are able to preserve both DNA and also RNA.

Advantageously, the preservation by means of the solution as above in accordance with the present description or the liquid solution obtained by dissolving the dry powder composition in accordance with the present description in the solvent does not provide the additional use of any pH regulator compounds whatsoever, such as acids, bases or buffer compounds.

Other embodiments concern a method of analysis for molecular diagnostics based on nucleic acids. This method provides to make available a biological sample from a swab which is preserved by means of immersion in the solution as above in accordance with the present description or in the liquid solution obtained by dissolving the dry powder composition in accordance with the present description in the solvent, and to carry out a technique of extracting and analyzing the nucleic acids on the preserved sample in order to detect the infection.

In one possible advantageous implementation, the sample as above is a salivary sample.

In possible embodiments, the diagnosis as above is aimed at detecting upper respiratory tract infections.

In one possible advantageous implementation, the diagnosis as above is aimed at detecting Coronavirus Disease 19 (COVID-19) caused by the Sars-CoV-2 virus.

In one possible implementation, the nucleic acids as above are RNA or DNA.

In one possible implementation, the analysis as above is qualitative and/or quantitative.

Another aspect of the present description is the use of a salivary sample, appropriately collected and preserved, for molecular diagnostics aimed at detecting infections.

According to one possible embodiment, this salivary sample is preserved immersed in a liquid aqueous solution according to the present description or in a liquid solution obtained by dissolving a dry powder composition according to the present description in a solvent.

In particular, according to possible embodiments, the infections as above are upper respiratory tract infections.

Even more in particular, according to possible embodiments, the infection as above is Coronavirus Disease 19 (COVID-19) caused by the Sars-CoV-2 virus.

### DESCRIPTION OF THE DRAWINGS

These and other aspects, characteristics and advantages of the present invention will become apparent from the following description of some embodiments, given as a non-restrictive example with reference to the attached drawings wherein:

FIGS. 1, 2 and 3 are a sequence of band intensity comparison graphs obtained by means of gel electrophoresis (of which images of example cases are reported) of amplified RNA or DNA, preserved with different preservative solutions in different time and temperature modes;

FIG. 4 shows images of bands obtained by means of gel electrophoresis of amplified RNA and DNA after freezing/thawing cycles;

FIG. 5 shows images of Petri dishes for bacterial culture on BPM medium (above) and Sabouraud (below) comparing the inhibitory activity on bacterial growth of the preservative solutions.

### DETAILED DESCRIPTION OF SOME EMBODIMENTS

We will now refer in detail to the possible embodiments of the invention, of which the experimental results are shown in the attached drawings. The phraseology and terminology used here is also for the purposes of providing nonlimiting examples.

Unless otherwise indicated, all measurements are carried out at 25° C. and at atmospheric pressure. Unless otherwise indicated, all temperatures are expressed in degrees Celsius.

All percentages and ratios indicated shall be understood to refer to the weight of the solution or total composition (w/w), unless otherwise indicated.

All percentage ranges reported here are provided with the provision that the sum with respect to the overall solution or composition is 100%, unless otherwise indicated.

All the intervals reported here shall be understood to include the extremes, including those that report an interval “between” two values, unless otherwise indicated.

The present description also includes the intervals that derive from overlapping or uniting two or more intervals described, unless otherwise indicated.

The present description also includes the intervals that can derive from the combination of two or more values taken at different points, unless otherwise indicated.

According to the invention, an aqueous solution is provided to stably preserve nucleic acids comprising:
an EDTA-based compound between 0.70 and 0.78% w/w with respect to the total weight of the solution;
ammonium sulphate between 17.5 and 38.5% w/w with respect to the total weight of the solution;
a sodium citrate based compound between 0.70 and 0.77% w/w with respect to the total weight of the solution.

The EDTA-based compound allows to chelate ions, in particular divalent ions, such as magnesium and calcium. By subtracting these ions from the solution, the activity of magnesium-dependent nucleases and calpain is inhibited. Furthermore, it minimizes contamination by metal ions which can adversely affect the purity and yield of subsequent reactions.

According to some embodiments, the EDTA-based compound is selected from a group comprising: EDTA, EDTA dihydrate disodium salt and EDTA tetrahydrate sodium salt. In a preferred embodiment, the EDTA-based compound is EDTA dihydrate disodium salt.

The ammonium sulfate allows the precipitation of proteins and the inactivation of nucleases and, at the same time, it promotes, by stabilizing them, the recovery of nucleic acids. It allows the extraction of amplifiable nucleic acids for molecular investigations.

The sodium citrate based compound supplies salts to the aqueous solution which have the function of stabilizing the structure of the nucleic acids, in particular DNA, reducing the electrostatic charges between the phosphate groups and promoting the step of extracting the nucleic acids.

The solvent is preferably purified deionized water suitable for molecular biology applications.

According to some embodiments, the sodium citrate based compound is selected from a group comprising: sodium citrate, sodium citrate dihydrate, monobasic sodium citrate, tribasic sodium citrate hydrate, tribasic sodium citrate dihydrate. In a preferred embodiment, the sodium citrate based compound is sodium citrate trisodium salt dihydrate.

The Applicant has experimented, in particular, aqueous solutions in which the EDTA compound is EDTA dihydrate disodium salt and the sodium citrate based compound is sodium citrate trisodium salt dihydrate.

The Applicant has also experimented aqueous solutions made by combining different w/w percentage ranges of EDTA dihydrate disodium salt, of ammonium sulphate, and of sodium citrate trisodium salt dihydrate, in order to obtain solutions suitable to effectively preserve both DNA and RNA.

In particular, the Applicant has identified three w/w percentage ranges defined as HIGH, MEDIUM, LOW as summarized in table 1.

With reference to the EDTA compound, the “LOW” range is between 0.56 and 0.63% w/w with respect to the total weight of the solution, preferably between 0.57 and 0.62%, preferably between 0.58 and 0.61%, more preferably between 0.59 and 0.60%;
the “MEDIUM” range is between 0.70 and 0.78% w/w with respect to the total weight of the solution, preferably between 0.71 and 0.77%, preferably between 0.72 and 0.76%, more preferably between 0.73 and 0.75%;
the “HIGH” range is between 0.86 and 0.93% w/w with respect to the total weight of the solution, preferably between 0.87 and 0.92%, preferably between 0.88 and 0.91%, more preferably between 0.89 and 0.90%.

With reference to the ammonium sulphate, the “LOW” range is between 17.5 and 24.5% w/w with respect to the total weight of the solution, preferably between 18.5 and 23.5%, preferably between 19.5 and 22.5%, more preferably between 20.5 and 21.5%;
the “MEDIUM” range is between 31.5 and 38.5% w/w with respect to the total weight of the solution, preferably between 32.5 and 37.5%, preferably between 33.5 and 36.5%, more preferably between 34.5 and 35.5%;
the “HIGH” range is between 66.5 and 73.5% w/w with respect to the total weight of the solution, preferably between 67.5 and 72.5%, preferably between 68.5 and 71.5%, more preferably between 69.5 and 70.5%.

With reference to the sodium citrate, the “LOW” range is between 0.55 and 0.62% w/w with respect to the total weight of the solution, preferably between 0.56 and 0.61%, preferably between 0.57 and 0.60%, more preferably between 0.58 and 0.59%;
the “MEDIUM” range is between 0.70 and 0.77% w/w with respect to the total weight of the solution, preferably between 0.71 and 0.76%, preferably between 0.72 and 0.75%, more preferably between 0.73 and 0.74%;
the “HIGH” range is between 0.85 and 0.92% w/w with respect to the total weight of the solution, preferably between 0.86 and 0.91%, preferably between 0.87 and 0.90%, more preferably between 0.88 and 0.89%.

The compounds used, and the w/w percentages, must not be considered as restrictive factors with regard to the field of protection, which is understood as expressed only by the attached claims.

TABLE 1

Percentage ranges of the compounds

| | EDTA dihydrate disodium salt (%w/w) | Ammonium Sulphate (%w/w) | Sodium citrate trisodium salt dihydrate (%w/w) |
|---|---|---|---|
| LOW | 0.56 - 0.63 | 17.5 - 24.5 | 0.55 - 0.62 |
| MEDIUM | 0.70 - 0.78 | 31.5 - 38.5 | 0.70 - 0.77 |
| HIGH | 0.86 - 0.93 | 66.5 - 73.5 | 0.85 - 0.92 |

The Applicant has identified two possible variants of aqueous solution suitable for the purposes, an aqueous solution 1 comprising EDTA dihydrate disodium salt, ammonium sulfate and sodium citrate trisodium salt dihydrate, each with a “MEDIUM” range, and an aqueous solution 2 which differs from the aqueous solution 1 in having ammonium sulphate with “LOW” range.

The experimental results obtained from the use of compositions 1 and 2 in the preservation of biological samples for subsequent molecular biology analyzes are provided as support hereafter in this description.

According to some embodiments, the pH of the aqueous solution is between 5.2 and 5.8, preferably between 5.3 and 5.7, more preferably between 5.4 and 5.7, even more preferably between 5.5 and 5.7.

In accordance with some embodiments, the pH of the aqueous solution obtained allows an advantageous preservation of both DNA and also RNA.

Advantageously, therefore, the liquid aqueous solution described here is without pH regulator compounds.

The aqueous solution examined by the Applicant allows to achieve one of the pH ranges described above without needing to correct the pH with the addition of acids or bases generally used for this purpose such as, for example, sulfuric acid or hydrochloric acid or sodium hydroxide.

Consequently, it is not necessary to have available environments suitable to store acids/bases and plants for their use, making the production process safer and more economical.

According to some embodiments, the liquid aqueous solution can comprise a surfactant between 0.01 and 0.1% w/w with respect to the weight of the solvent.

Example percentages of surfactant can be 0.01%, 0.015%, 0.02%, 0.025%, 0.03%, 0.035%, 0.04%, 0.045%, 0.05%, 0.055%, 0.06%, 0.065%, 0.07%, 0.075%, 0.08%, 0.085%, 0.09%, 0.095% or 0.1%.

The surfactant is provided to solubilize cell membranes, produce protein complexes and facilitate the extraction of nucleic acids. It can also make a possible treatment with proteinases, for example proteinase K, more efficient by exposing the cleavage sites of the proteins to be degraded.

In preferred embodiments, the surfactant is an anionic surfactant, more preferably Sodium Dodecyl Sulfate (SDS). Preferably, the w/w percentage of SDS is between 0.02 and 0.08%, preferably between 0.03 and 0.07%, even more preferably between 0.04 and 0.06%. Example percentages of SDS can be 0.02%, 0.025%, 0.03%, 0.035%, 0.04%, 0.045%, 0.05%, 0.055%, 0.06%, 0.065%, 0.07%, 0.075%, 0.08%.

In some embodiments, a dry powder composition suitable to be dissolved in a solvent in order to provide an aqueous solution in accordance with some embodiments described above can be provided.

In accordance with some embodiments, the dry powder composition comprises at least:
an EDTA-based compound between 1.9 and 3.8% w/w with respect to the total weight of the composition;
ammonium sulfate between 92.5 and 96.2% w/w with respect to the total weight of the composition;
a sodium citrate based compound between 1.9 and 3.7% w/w with respect to the total weight of the composition.

The dry composition thus provided has to be dissolved in an appropriate quantity of deionized water in order to reconstitute the aqueous solution in liquid form with the concentration characteristics described in claim 1.

The dry powder composition according to the present description is also free of pH regulator compounds and, when it is dissolved in a polar solvent, no pH-regulator compound is added to it.

In order to make solution 1, a dry powder composition is provided in which the EDTA-based compound is between 2.1 and 2.5% w/w, the ammonium sulfate is between 95 and 96% w/w and the sodium citrate based compound is between 2.1 and 2.5% w/w.

In order to make solution 2, a dry powder composition is provided in which the EDTA compound is between 2.7 and 3.7% w/w, the ammonium sulfate is between 92 and 95% w/w and the sodium citrate based compound is between 2.7 and 3.7% w/w.

The invention also concerns a containing device, for example a test tube or a vial, comprising the aqueous solution or the dry powder composition in accordance with the embodiments described here.

In some embodiments, the containing device can be suitable to at least partly accommodate inside it a collection member configured to perform any type of collection whatsoever, for example by means of a swab but also by means of other collection and sampling modes. The collection member comprises at least one collection end configured to contact the biological material.

The aqueous solution is supplied with a volume suitable to allow the immersion of the biological sample, so that it is correctly in contact with the aqueous solution. For example, in the case of collection performed by means of a swab, the suitable volume of the containing device is able to accommodate at least the sample collection end.

In other embodiments, if the containing device comprises the dry powder composition, the latter is supplied with a weight suitable to be dissolved in a volume of solvent sufficient to immerse and cover the sample collected.

The invention also concerns a kit for collecting and preserving a biological sample comprising the containing device and a collection member.

Furthermore, another aspect of the present description is a method to preserve nucleic acids comprising making available a liquid aqueous solution in accordance with the present description or a dry powder composition in accordance with the present description, suitably dissolved in a solvent, and introducing and keeping immersed a biological sample in such liquid aqueous solution or in the solution obtained by dissolving the dry powder composition as above in the solvent.

Advantageously, the preservation by means of the aqueous solution as above in accordance with the present description or the liquid solution obtained by dissolving the dry powder composition in accordance with the present description in the solvent does not provide the additional use of any pH regulator compound whatsoever, such as acids, bases or buffer compounds.

Another aspect of the present disclosure is a method of analysis for molecular diagnostics based on nucleic acids. This method provides to make available a biological sample from a swab which is preserved by means of immersion in the solution as above in accordance with the present description or in the liquid solution obtained by dissolving the dry powder composition in accordance with the present description in the solvent, and to carry out a technique for extracting and analyzing the nucleic acids on the preserved sample in order to detect the infection.

Advantageously, the sample to be preserved and analyzed, and the nucleic acids contained therein, are supplied by means of a swab and in particular, advantageously, by means of a salivary sample.

The nucleic acids which can be preserved in accordance with the present description and subsequently subjected to analysis can be both DNA and also RNA. Unlike the state of the art, in fact, the present invention allows the preservation for the purposes of the subsequent analysis of both types of nucleic acids and possibly also of other types of nucleic acids.

Favorably, the diagnosis as above is aimed at detecting upper respiratory tract infections. In one advantageous example, the diagnosis as above is aimed at detecting Coronavirus Disease 19 (COVID-19) caused by the Sars-CoV-2 virus.

The present invention therefore makes available a saline solution, as such or obtainable by dissolving the dry powder composition described here, to transport and preserve salivary samples and nasopharyngeal swabs, which allows to carry out the molecular determination of viral RNA.

The solution that can be obtained according to the present invention has an acid pH which is optimal for stabilizing human and potentially also animal, viral and bacterial nucleic acids (DNA, RNA). The high salt concentration and ionic charge determine a denaturation of the proteins and enzymes, in particular of nucleases (RNases) and an inactivation of cell proliferation, unlike common commercial transport media.

Another advantage of the solution that can be obtained according to the present description is represented by the ability to fluidify saliva, making it suitable for subsequent molecular investigations. In fact, as it appears, saliva would not be adequate for RT-PCR tests, for example for the search for the SARS-CoV-2 viral genome.

### EXPERIMENTAL EXAMPLES

The experimental examples described below were designed and implemented to evaluate the suitability of the two variants of aqueous solution (aqueous solution 1 and aqueous solution 2) for preserving DNA and RNA. In order to judge the preservation capacity, the aqueous solutions 1 (S1) and 2 (S2) were compared with known preserving solutions, in particular RNAlater (R) used to preserve nucleic acids, UTM - COPAN (C) and a solution of HBSS (H) added with 2% w/w of heat-inactivated fetal bovine serum and gentamicin at a final concentration of 50 mg/mL, used for maintaining the vitality of microorganisms in a biological sample that has to be analyzed with molecular diagnostic techniques.

The ability of the saline solutions 1 and 2 to stabilize and maintain the nucleic acids intact even after repeated freezing and thawing cycles, and the ability to inhibit bacterial growth were also evaluated.

In the images of the electrophoretic gels, where provided, of the accompanying tables, MW are the molecular weights, NEG is the negative control (absence of nucleic acids), POS is the positive control (presence of nucleic acids).

### 1. Collection and Preservation of the Biological Samples

The collection of the biological samples was carried out by performing a buccal swab using a collection member, in order to simulate a normal diagnostic procedure.

The samples collected were preserved in sets of five test tubes, each containing a different preserving solution, in this specific case: HBSS, RNAlater, the aqueous solution 1 (S1), the aqueous solution 2 (S2) and COPAN.

In order to evaluate the effect of the preservation temperatures and time, each set of test tubes containing the biological sample was preserved at room temperature (RT) or at 4° C. or at -20° C. for the following times: 24 h, 48 h and 7 days.

Before preservation, approximately 100,000 pathogenic bacteria cells (St. aureus) were inserted into each test tube in order to simulate a bacterial load.

### 2. Extraction of the Nucleic Acids and PCR Assays

The preservation of the nucleic acids was evaluated by amplification of gene products or ribosomal RNA with end-point PCR and visualization of the amplified products on electrophoretic gel.

Several nucleic acid extraction strategies were tested:
Total RNA Purification Kit (Norgen), recommended for buccal swabs, in particular for the extraction of RNA;
Quick-DNA Miniprep Kit (Zymo Research) for the extraction of DNA;
Trizol (Qiagen) for the extraction of RNA.

The presence and integrity of the following was verified with end-point PCRs:
a) 18 s ribosomal RNA;
b) Human beta-actin
c) Bacterial gene
d) DNA fragments (multiplex with 4 target genes with amplicons of different sizes) for the qualitative analysis of genomic integrity.

The amplifications of each end-point PCR were loaded on wells of an agarose gel obtained from a solution with a suitable buffer with 2% weight/volume with respect to the total of the agar solution and a double filament DNA and/or cDNA intercalating agent, for example SYBR Green or SYBR Safe. The quantitative analysis of the bands obtained was performed with the software Image J.

### 3. Results

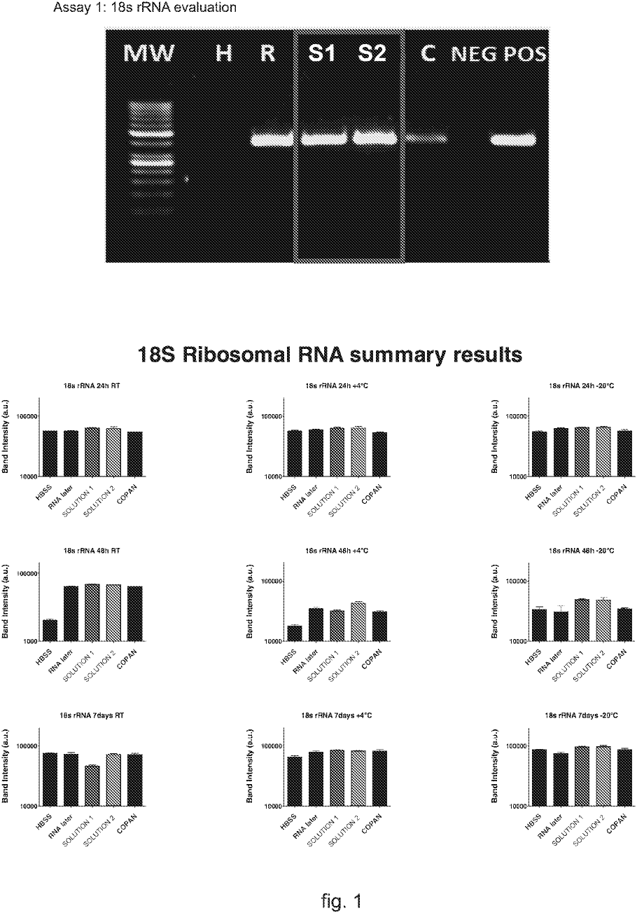

With reference to FIGS. 1, 2 and 3, these show a sequence of histograms obtained by acquiring the intensity of the bands of the end-point PCR products from the agarose gel for the conditions examined by the experimental design, available for reference in paragraph 2 “Collection and preservation of the biological samples”.

With reference to FIG. 1, this shows the results relating to the amplification of 18 s ribosomal RNA.

From these results it can be seen that at 24 h the liquid solution has a preservation efficiency substantially similar to that achieved with the known solutions tested here.

At 48 h, and for all the times taken into consideration, the liquid solution provided by the invention is significantly more efficient than the HBSS composition. Compared to specific solutions such as RNAlater and HBSS, in particular in the condition at 4° C. and -20° C., the liquid solution provided by the invention has a comparable or even higher preservation of 18 s ribosomal RNA.

This result is particularly significant given that molecular analysis is usually performed within this time window and, therefore, a high degree of preservation is required.

At seven days, the liquid solution has a preservation capacity substantially comparable to the other tested solutions.

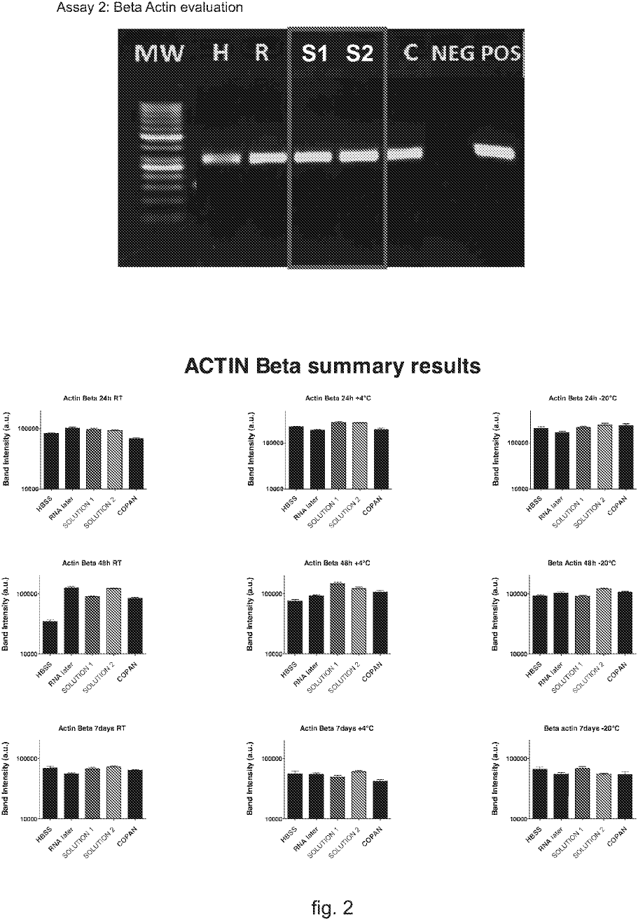

With reference to FIG. 2, this shows the results relating to the amplification of a portion of the Beta-Actin gene.

The results obtained demonstrate that the liquid solution preserves DNA in a manner comparable or superior to the other tested solutions.

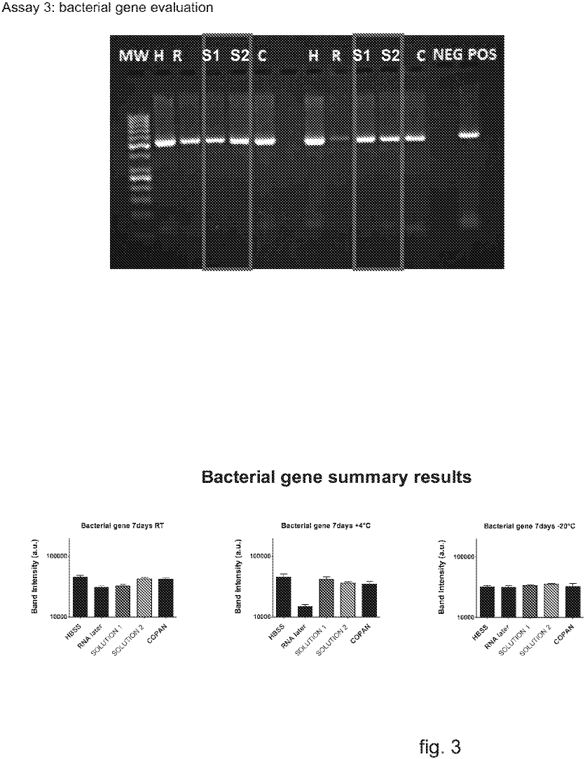

With reference to FIG. 3, in which the amplification of a bacterial gene of St. aureus is evaluated after seven days after collection, this demonstrates how the liquid solution has the ability to preserve DNA in a manner comparable or superior to the other tested solutions.

A known factor that negatively affects the integrity of nucleic acids is the repeated freezing/thawing of the biological sample.

In order to evaluate the preservation capacity of the liquid solution under these conditions, some biological samples were subjected to three freezing/thawing cycles and analyzed ten days after collection.

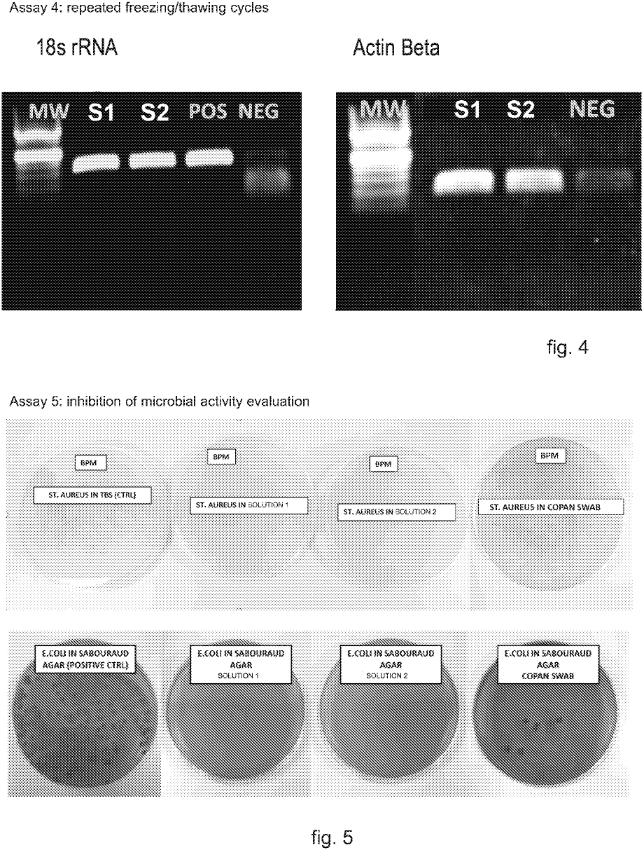

As shown in FIG. 4, even after the repeated freezing/thawing cycles, it is still possible to obtain an amplification both from 18 s ribosomal RNA and also from DNA sequence (Beta-Actin).

In accordance with the purposes of the invention, the ability of the liquid solution to inhibit bacterial growth was evaluated.

After about three hours after collection and the addition of St. aureus cells, 100 µL of sample were sown on Baird Parker Medium (BPM) and Sabouraud Agar microbial growth medium. Bacterial growth was evaluated after 30 h of culture.

As shown in FIG. 5, the Petri dishes in which the sample preserved in the liquid solution was sown did not show any bacterial growth. It should be noted that this result is achieved without the presence of antibiotics in the medium, but simply with the specific combination of salts identified by the Applicant. The result is surprising when compared with the result obtained with UTM-COPAN which, although provided with an antibiotic, did not effectively inhibit bacterial growth.

The same analysis was performed by the Applicant also with E. Coli with similar results.

### IN VIVO EXPERIMENTAL DATA ON MOLECULAR TEST CONDUCTED ON SALIVARY SAMPLE AND NASOPHARYNGEAL SWAB

The Applicant has tested a method that provides collection of a sample of saliva or collection from a nasopharyngeal swab, transport of the sample in saline aqueous solution in accordance with the present description and a molecular test for the analysis of RNA of SARS-CoV-2 virus, in terms of accuracy compared to the test on gold-standard nasopharyngeal swab collected in COPAN medium.

Furthermore, the Applicant has estimated the sensitivity and specificity of the method applied to the sampling collected in the aqueous solution and evaluated the characteristics of the method in terms of limit of detection (LoD), linearity and precision.

### Experimental Design

In the preliminary phase, the collection system using the aqueous solution according to the present description was validated on different automated or manual extraction systems using 10 salivary samples and 10 nasopharyngeal swabs which were compared with the respective nasopharyngeal swabs collected in COPAN medium. The technologies tested use magnetic beads, silica membrane or resin combined with column chromatography. The tests for compatibility with the extraction procedures were carried out within 24 hours of collection. As regards the methods of amplification and detection of nucleic acids, the aqueous solution according to the present description has proven to be compatible with real time RT-PCR, Droplet Digital PCR (ddPCR) system, Xpert Xpress SARS-CoV-2 system, Cepheid, Panther system (Hologic) and Next Generation Sequencing (NGS).

In order to verify the ability to keep viral RNA stable in saliva, we compared the results obtained from 25 freshly collected salivary samples, without preservative solutions, with the respective salivary samples collected in the aqueous solution according to this description, obtained from the same patient.

The detailed procedure systematically used throughout the entire validation phase performed on 1076 samples will be described below.

### Participants and Sample Collection

This prospective study has allowed to analyze the salivary samples of 161 patients (age ≥18 years) with COVID-19 infection and 915 subjects belonging to a group of asymptomatic controls tested in the screening phase. A double sample collection, on a sample of 95 patients, was carried out using a nasopharyngeal swab in order to compare the effect of the two transport means, that is, aqueous solution according to the present description compared to COPAN medium, on the result of the molecular test.

All samples collected were made anonymous using an alphanumeric identification code. Informed consent was requested from the participants by means of a specific form.

The samples were collected in 1.5 mL of aqueous solution according to the present description.

In the case of salivary samples, the patient placed the sterile collection tube on his/her lower lip and let a volume of saliva equal to that of the solution flow into the test tube. The test provided the simultaneous collection of the nasopharyngeal swab with gold standard COPAN medium and the saliva sample, in order to compare the performance of the two sampling techniques on the subsequent molecular analysis. Similarly, in order to compare the stabilizing capacity of the two transport solutions (COPAN medium and aqueous solution according to the present description), two samples of the upper respiratory tracts were collected in parallel by means of a nasopharyngeal swab.

### Extraction of the Viral RNA

The viral RNA was isolated starting from 800 µL of saliva by means of automated extraction on Qiasymphony instrumentation (Qiagen) using the Virus/Pathogen kit (Qiagen).

### Analysis by Means of RT-PCR

Real-time RT-PCR was used for the detection of gene E, which encodes the viral envelope protein of SARS-CoV-2 (COVID-19). The beta actin gene was employed as an endogenous control to evaluate the efficiency of RNA extraction. The primer sequences used for the amplification reaction were selected according to the guidelines of the CDC and purchased from Roche (Roche); the gene E specific probe is conjugated with the fluorophore FAM, while the one for beta actin with HEX. Amplification was performed using LightCycler® Multiplex RNA Virus Master (Roche) master mix, following the company’s instructions. For each reaction, 5µL of previously extracted viral RNA were loaded. For each reaction, a synthetic RNA positive control and a no template control were used.

The thermal conditions of amplification were: 15 minutes at 55° C. for the reverse transcription reaction; 5 minutes at 95° C. for the activation of the Taq-polymerase; 1 minute at 94° C. and 1 minute at 60° C. (for 45 cycles) for the actual amplification.

The PCR and analyzes were performed using the instrument LightCycler 480 (Roche). For each amplification reaction the Ct (Cycle threshold) values were calculated. The Ct value is inversely correlated to the viral load and each increase in Ct of approximately 3.3 reflects a 10-fold reduction in the starting material. The positivity cut-off is established with Ct equal to 36, which corresponds to the detection of 5 starting RNA molecules.

### Quantification of the Absolute Number of Viral Copies

The absolute number of viral copies was first assessed by constructing a standard curve based on RNA of a known type. The amplification efficiency, calculated starting from the calibration line, had as reference values a correlation coefficient R² (determination index) equal to 0.99 and a slope equal to -0.3.

The calculation of the number of viral copies present in the salivary and nasopharyngeal samples collected was performed through the trend function in which the Ct value associated with each point of the calibration curve was interpolated with the logarithmic value of the number of copies, so as to obtain the linear interpolation line.

However, using RT-PCR may have some limitations in quantifying extremely low concentrations of viral copies. To overcome this limitation, another absolute quantification of the collected samples was performed by means of droplet digital PCR.

Droplet digital PCR (ddPCR) represents a latest generation molecular approach with theoretical sensitivity equal to 0.0001% (1 copy out of 10,000) and with high performance in terms of applicability and sensitivity.

The principle behind ddPCR is to perform an absolute quantification of the target nucleic acid present in a sample, distributing it randomly within partitions (droplets), so that a copy of the target is individually amplified during PCR.

ddPCR allows to overcome some of the limitations of traditional real-time qPCR, improving its sensitivity and reproducibility. The viral load present in the saliva samples was quantified in an absolute manner using One-Step RT-ddPCR Advanced Kit for Probes (BioRad).

The reaction mix comprises a mixture of primer and probe specific for the FAM-conjugated SARS-CoV2 E gene (900 nM and 250 nM primers) and for the HEX-conjugated beta actin gene required to assess the cellularity of the sample. Droplet generation was performed using QX200 (BioRad).

The conditions of the thermal cycle used were: 50° C. for 1 hour (reverse transcription); 95° C. for 10 min; and 45 cycles of 95° C. for 30 sec and 55° C. for 1 min. The results were analyzed on a QX200 reader (BioRad) using Quanta Soft software (BioRad). Positive droplets, containing amplification products, were discriminated from the negative ones by applying a fluorescence amplitude threshold.

### Statistical Analysis

The level of agreement was measured using Cohen’s kappa method. The sensitivity and specificity of the method applied to the salivary collection were estimated with the corresponding confidence interval.

Positive percentage agreement (PPA) was calculated as the percentage of positive individuals identified by the gold standard (nasopharyngeal swab) and who also tested positive in the salivary test. Similarly, negative percentage agreement (NPA) was expressed as the proportion of individuals who tested negative in the nasopharyngeal swab, who tested negative in the salivary test.

The Limit of Detection (LoD) was evaluated with 5 serial dilutions (10 times each) of viral RNA isolated from a positive patient by the internal method and evaluated by RT-qPCR. Each standard point was analyzed in 10 replicates. Data distribution was checked with the Shapiro-Wilk test.

The linearity of the internal method was established with 7 serial dilutions of viral RNA evaluated by RT-qPCR.

Repeatability was assessed by testing a positive sample on 3 consecutive days, repeating the extraction procedure at the same time of the day. Reproducibility (intermediate precision) was evaluated by analyzing the same sample 3 times at 3 different concentrations. Intermediate precision was evaluated with repeated measures ANOVA, according to ISO 5725 guidelines.

### Validation Results

### Comparison With the Nasopharyngeal Swab

### I) Sensitivity and Specificity

For the validation of the method, 161 salivary samples of a population of symptomatic patients with COVID-19 infection and 915 samples of asymptomatic subjects tested in the screening phase were analyzed, for an overall total of 1076 subjects. A nasopharyngeal swab was also performed on 95 of the 161 symptomatic subjects, which was then collected in the liquid aqueous solution described here. The results of the salivary molecular test (SALIVA) and the nasopharyngeal swab (NF SWAB) collected in the solution described here were compared with those obtained with the nasopharyngeal swab collected in COPAN medium, gold-standard reference. 123 subjects tested positive in the salivary test, compared to 104 identified by the nasopharyngeal swab with COPAN medium. 24 subjects tested positive in the salivary test and negative in the nasopharyngeal swab collected with COPAN medium. The detailed results, regarding the salivary samples, are presented in Tables 2 and 3 below:

TABLE 2

Number and results of molecular tests performed on nasopharyngeal swab and saliva

| | SALIVA neg | SALIVA pos | Tot |
|---|---|---|---|
| NF SWAB neg | 948 | 24 | 972 |
| NF SWAB pos | 5 | 99 | 104 |
| Tot | 953 | 123 | 1076 |

TABLE 3

Number and results of the molecular tests obtained and divided into the two populations in question, discriminated on the basis of the presence/absence of the disease.

| | SALIVA neg | SALIVA pos | Tot |
|---|---|---|---|
| SICK | 5 | 99 | 104 |
| CONTROLS | 948 | 24 | 972 |
| Tot | 953 | 123 | 1076 |

Taking as a reference the subjects with pathology (M) and with a positive nasopharyngeal test (+), the clinical sensitivity achieved by the salivary molecular test was 95.2% while the calculated specificity was 97.5%. The details relating to the variation in sensitivity and specificity levels in relation to the prevalence of the disease are shown in Table 4.

TABLE 4

Calculated sensitivity and specificity. Pr(M) = prevalence of the disease, M=sick; C=controls; +=positive test; -=negative test

| | | [Confidence interval 95%] | | |
|---|---|---|---|---|
| Prevalence | Pr(M) | 9.7% | 8% | 11.6% |
| Sensitivity | Pr(+/M) | 95.2% | 89.1% | 98.4% |
| Specificity | Pr(-/C) | 97.5% | 96.3% | 98.4% |

As regards the results of the molecular tests performed on the 95 nasopharyngeal swabs collected in the liquid aqueous solution in accordance with the present description (NF SWAB SOLUTION) compared to the COPAN medium (NF SWAB COPAN), the following results were obtained, presented in Tables 5 and 6:

TABLE 5

Number and results of molecular tests performed on nasopharyngeal swab collected in the liquid solution in accordance with the present description compared with COPAN medium

| | NF SWAB SOLUTION neg | NF SWAB SOLUTION pos | Tot |
|---|---|---|---|
| NF SWAB COPAN neg | 49 | 9 | 58 |
| NF SWAB COPAN pos | 9 | 28 | 37 |
| Tot | 58 | 37 | 95 |

TABLE 6

Number and results of molecular tests obtained and divided on the basis of the test result obtained on the gold-standard

| | NF SWAB SOLUTION neg | NF SWAB SOLUTION pos | Tot |
|---|---|---|---|
| SICK (TEST +) | 9 | 28 | 37 |
| CONTROLS (TEST -) | 49 | 9 | 58 |
| Tot | 58 | 37 | 95 |

Table 7 below presents the results of the calculation of clinical sensitivity and specificity, with the premise that the study was conducted on the population of symptomatic subjects only. The calculation was made considering as a reference the result of the molecular test performed on a nasopharyngeal swab with COPAN medium.

TABLE 7

Calculated sensitivity and specificity. Pr(M)=prevalence of the disease, M=sick; C=controls; +=positive test; -=negative test

| | | |
|---|---|---|
| Prevalence | Pr(M) | 61% |
| Sensitivity | Pr(+/M) | 84.5% |
| Specificity | Pr(-/C) | 75.6% |

Also in this case, we can confirm that the test carried out on saliva reaches a level of clinical sensitivity 11% higher than the test performed on a nasopharyngeal swab, regardless of whether the latter is collected in COPAN medium or in liquid aqueous solution according to the present description. The increase in clinical sensitivity is in fact determined by the greater cellularity of the salivary sample compared to the nasopharyngeal collection.

### II) Agreement: Cohen’s Kappa

In order to measure the agreement between the results of the salivary test, compared to the nasopharyngeal swab, Cohen’s Kappa was calculated, which resulted in:

$$\text{Kappa} \left(95 \% \text{CI}\right) = 0.857 \left(0.806 \text{-} \text{0} \text{.908}\right)$$

The agreement reached is equal to 86%, with an accuracy of 5%. The Cohen’s Kappa reached, comprised in the range 0.81-1.00, indicates an optimal level of agreement between the two methods compared, in accordance with Julia Alcoba-Florez et al. Sensitivity of different RT-qPCR solutions for SARS-CoV-2 detection. Int J Infect Dis. 2020.

As for Cohen’s Kappa obtained between molecular tests performed on nasopharyngeal swab collected in BSWAB and nasopharyngeal swab collected in COPAN, the result was:

$$\text{Kappa} \left(95 \% \text{CI}\right) = 0.602 \left(0.61 \text{-0} \text{.80}\right)$$

The agreement reached is equal to 81%, with an accuracy of 5%. The Cohen’s Kappa reached, comprised in the range 0.61-0.80, indicates a good level of agreement between the two methods compared, in accordance with McHugh ML. Interrater reliability: the kappa statistic. Biochem Med (Zagreb). 2012;22(3)276-282.

### II) Agreement Between the Two Methods

The agreement reached between the salivary test and the COPAN nasopharyngeal swab was 97.3% (Std Error=0.030; Z=28.25; Prob>Z=0.000).

The agreement reached between the results of the molecular test carried out on nasopharyngeal swab in the liquid aqueous solution according to the present description compared to the COPAN medium was 81.1% (Std Error=0.030; Z=28.25; Prob>=0.000).

### Stability

The stability of the salivary samples was performed on 72 samples, verifying the consistency of the results obtained both in RT PCR and also in ddPCR at t₀ (time of collection), at t₁ (24 h from collection) and at t₂ (48 h from collection). Two preservation temperatures were also tested at each time-point: room temperature and sample refrigeration at 4° C.

There was no loss of positivity in any of the time/temperature combinations, and variations in Ct or number of copies were not significant.

### Limit of Detection (LOD) and Repeatability

The limit of detection (LOD) of the salivary sample was determined both by RT-PCR and also by ddPCR measuring the correlation between the Ct value and the number of copies detected.

The minimum number of identifiable viral copies is equal to 5 and corresponds to a Ct value of 36.7 ± 0.27.

The mean number of viral copies measured in the salivary samples was 50,000 copies against the 12,500 quantified in the nasopharyngeal swabs. Both measurements refer to 5 µL of extract analyzed by RT-PCR.

### Comments on the Experimental Data

The overall results obtained allowed to validate the procedure for collection, transport in the liquid aqueous solution of the present description of salivary samples and nasopharyngeal swabs for subsequent molecular investigation to diagnose SARS-CoV-2.

The method has proven to be accurate, specific and sensitive, especially when applied to salivary samples. The extension of the diagnostic procedure to salivary samples offers the possibility of extending the test to fragile subjects such as the elderly, children and uncooperative subjects, and to free specialized health personnel, since the test can be self-administered.

Therefore, the liquid aqueous solution of the present description has proven effective in stabilizing the viral genome up to the moment of molecular analysis, offering an alternative and more performing tool for the collection of biological samples to be subjected to tests that research nucleic acids, with a diagnostic intent.

The liquid aqueous solution of the present description is able to fluidify saliva making it a matrix which can be potentially used for all molecular tests, even those other than COVID-19 research.

Its high salt concentration and ionic strength cause proteins to be denatured resulting in cell breakdown. As with other solutions with high salt concentrations, it has a bacteriostatic effect and inhibits cell proliferation, which could be a point of strength in the collection of infectious samples.

Furthermore, the liquid aqueous solution that can be obtained according to the embodiments described here is advantageous since it has a stabilized pH without needing to add pH regulator compounds, such as acids, bases or buffer compounds.

It is clear that modifications may be made to the liquid aqueous solution as described heretofore, without departing from the field and scope of the present invention as defined by the claims.

1. Aqueous solution to stably preserve nucleic acids comprising:
an EDTA-based compound between 0.70 and 0.78% w/w with respect to the total weight of the solution;
ammonium sulphate between 17.5 and 38.5% w/w with respect to the total weight of the solution;
a sodium citrate based compound between 0.70 and 0.77% w/w with respect to the total weight of the solution.

2. Aqueous solution as in claim 1, wherein said EDTA-based compound is selected from a group comprising: EDTA, EDTA dihydrate disodium salt and EDTA tetrahydrate sodium salt.

3. Aqueous solution as in claim 1, wherein said sodium citrate based compound is selected from a group comprising: sodium citrate, sodium citrate dihydrate, monobasic sodium citrate, tribasic sodium citrate hydrate, tribasic sodium citrate dihydrate.

4. Aqueous solution as in any claim from 1 to 3, wherein said aqueous solution comprises a surfactant between 0.01 and 0.1% w/w with respect to the total weight of the solution.

5. Aqueous solution as in claim 4, wherein said surfactant is preferably an anionic surfactant, even more preferably Sodium Dodecyl Sulfate (SDS) between 0.02 and 0.08% w/w with respect to the total weight of the solution.

6. Aqueous solution as in any claim from 1 to 5, wherein the pH of said aqueous solution is between 5.2 and 5.8, preferably between 5.3 and 5.7, more preferably between 5.4 and 5.7, even more preferably between 5.5 and 5.7.

7. Aqueous solution as in any claim from 1 to 6, wherein said aqueous solution is free of additional pH regulator compounds.

8. Dry powder composition suitable to be dissolved in a polar solvent, in particular water, comprising:
an EDTA-based compound between 1.9 and 3.8% w/w with respect to the total weight of the composition;
ammonium sulphate between 92.5 and 96.2% w/w with respect to the total weight of the composition;
a sodium citrate based compound between 1.9 and 3.7% w/w with respect to the total weight of the solution.

9. Dry powder composition as in claim 8, wherein said EDTA-based compound is selected from a group comprising: EDTA, EDTA dihydrate disodium salt and EDTA tetrahydrate sodium salt.

10. Dry powder composition as in claim 8, wherein said sodium citrate based compound is selected from a group comprising: sodium citrate, sodium citrate dihydrate, monobasic sodium citrate, tribasic sodium citrate hydrate, tribasic sodium citrate dihydrate.

11. Dry powder composition, wherein said dry powder composition is in itself free of pH regulator compounds and, when dissolved in said polar solvent, no pH regulator compound whatsoever is added to it.

12. Containing device for a biological sample comprising the aqueous solution as in any claim from 1 to 7 or the dry composition from 8 to 11.

13. Kit for collection and storage of nucleic acids, comprising a containing device as in claim 10 and a collection member provided at one end of a swab.

14. Kit as in claim 13, wherein said kit comprises deionized water to dissolve the dry powder composition.

15. Kit as in claim 13 or 14, wherein said kit comprises a proteinase K solution.

16. Method to preserve nucleic acids comprising making available the liquid solution as in any claim from 1 to 7 or the dry powder composition as in any claim from 8 to 11 dissolved in a solvent, and introducing and keeping immersed said biological sample in said liquid solution as in any claim from 1 to 7 or in said dry powder composition as in any claim from 8 to 11 dissolved in said solvent.

17. Method as in claim 16, wherein said nucleic acids are supplied by means of a swab.

18. Method as in claim 16, wherein said nucleic acids are provided by means of a salivary sample.

19. Method as in claim 17 or 18, wherein said liquid solution as in any claim from 1 to 7 or the liquid solution obtained by dissolving said dry powder composition as in any claim from 8 to 11 in said solvent are able to preserve both DNA and also RNA.

20. Method as in claim 16, 17 or 18, wherein the preservation by means of said liquid solution as in any claim from 1 to 7 or the liquid solution obtained by dissolving said dry powder composition as in any claim from 8 to 11 does not provide the additional use of any pH regulator compound whatsoever.

21. Method of analysis for molecular diagnostics based on nucleic acids, said method providing to make available a biological sample from a swab which is preserved by means of immersion in a liquid solution as in any claim from 1 to 7 or in a liquid solution obtained by dissolving a dry powder composition as in any claim from 8 to 11 in a solvent, and to carry out a technique for extracting and analyzing said nucleic acids on said preserved sample in order to detect said infection.

22. Method as in claim 21, wherein said sample is a salivary sample.

23. Method as in claim 21 or 22, wherein said diagnosis is aimed at detecting upper respiratory tract infections.

24. Method as in claim 23, wherein said diagnosis is aimed at detecting Coronavirus Disease 19 (COVID-19) caused by the Sars-CoV-2 virus.

25. Method as in any claim from 21 to 24, wherein said nucleic acids are RNA or DNA.

26. Method as in any claim from 20 to 25, wherein said analysis is qualitative and/or quantitative.

27. Use of a salivary sample for molecular diagnostics aimed at detecting infections.

28. Use as in claim 27, wherein said salivary sample is preserved immersed in a liquid solution as in any claim from 1 to 7or in a liquid solution obtained by dissolving a dry powder composition as in any claim from 9 to 11 in a solvent.

29. Use as in claim 27 or 28, wherein said infections are upper respiratory tract infections.

30. Use as in claim 27, 28 or 29, wherein said infection is Coronavirus Disease 19 (COVID-19) caused by the Sars-CoV-2 virus.

31. Method to prepare an aqueous solution to stably preserve nucleic acids, said method comprising making an aqueous solution containing:
an EDTA-based compound between 0.70 and 0.78% w/w with respect to the total weight of the solution;
ammonium sulphate between 17.5 and 38.5% w/w with respect to the total weight of the solution;
a sodium citrate based compound between 0.70 and 0.77% w/w with respect to the total weight of the solution.

32. Method as in claim 31, wherein said method does not provide to use or add any pH regulator compound whatsoever to said aqueous solution.
